SEQUENCE LISTING (12 sequences)

SEQ ID NO: 1 - protein, 92 aa, Homo sapiens
EEIGANDDLNYWSDWYDSDQIKEELPEPFEHLLQRIARRPKPQQFFGLMGKRDADSSIEKQVALLKALYGHGQISHKMAYERSAMQNYERRR

SEQ ID NO: 2 - protein, 110 aa, Homo sapiens
EEIGANDDLNYWSDWYDSDQIKEELPEPFEHLLQRIARRPKPQQFFGLMGKRDADSSIEKQVALLKALYGHGQISHKRHKTDSFVGLMGKRALNSVAYERSAMQNYERRR

SEQ ID NO: 3 - protein, 98 aa, Homo sapiens
EEIGANDDLNYWSDWYDSDQIKEELPEPFEHLLQRIARRPKPQQFFGLMGKRDAGHGQISHKRHKTDSFVGLMGKRALNSVAYERSAMQNYERRRSEQ

SEQ ID NO: 4 - protein, 77 aa, Homo sapiens
EEIGANDDLNYWSDWYDSDQIKEELPEPFEHLLQRIARRPKPQQFFGLMGKRDAGHGQISHKMAYERSAMQNYERRR

SEQ ID NO: 5 - protein, 37 aa, Homo sapiens
EEIGANDDLNYWSDWYDSDQIKEELPEPFEHLLQRIA

SEQ ID NO: 6 - protein, 11 aa, Homo sapiens
RPKPQQFFGLM

SEQ ID NO: 7 - protein, 36 aa, Homo sapiens
DADSSIEKQVALLKALYGHGQISHKRHKTDSFVGLM

SEQ ID NO: 8 - protein, 19 aa, Homo sapiens
GHGQISHKRHKTDSFVGLM

SEQ ID NO: 9 - protein, 10 aa, Homo sapiens
HKTDSFVGLM

SEQ ID NO: 10 - protein, 16 aa, Homo sapiens
ALNSVAYERSAMQNYE

SEQ ID NO: 11 - protein, 19 aa, Homo sapiens
GANDDLNYWSDWYDSDQIK

SEQ ID NO: 12 - protein, 16 aa, Homo sapiens
IKEELPEPFEHLLQRI

(19) United States
(12) Patent Application Publication
(10) Pub. No.: US 2024/0272178 A1
(43) Pub. Date: Aug. 15, 2024

(54) A METHOD FOR PREDICTING SEPSIS AND SEPTIC SHOCK

(71) Applicant: SphingoTec GmbH, Hennigsdorf (DE)

(72) Inventor: Andreas BERGMANN, Berlin (DE)

(73) Assignee: SphingoTec GmbH, Hennigsdorf (DE)

(21) Appl. No.: 18/568,334

(22) PCT Filed: Jun. 17, 2022
(86) PCT No.: PCT/EP2022/066592

§ 371 Date: Dec. 8, 2023

(30) Foreign Application Priority Data
Jun. 18, 2021 (EP) 21180359.8

(51) Int. Cl. G01N 33/68

(52) U.S. Cl. CPC G01N 33/6893; G01N 2800/26

(57) ABSTRACT

Subject matter of the present invention is a method for predicting sepsis, severe sepsis or septic shock in a patient comprising determining the level of Pro-Tachykinin A or fragments thereof of at least 5 amino acids in a bodily fluid obtained from said subject, and correlating the determined level of Pro-Tachykinin A or fragments thereof of at least 5 amino acids with sepsis or septic shock, wherein an elevated level above a certain threshold is predictive of sepsis, severe sepsis or septic shock.

Subject matter of the present invention is a method for predicting sepsis, severe sepsis or septic shock in a patient comprising determining the level of Pro-Tachykinin A or fragments thereof of at least 5 amino acids in a bodily fluid obtained from said subject, and correlating the determined level of Pro-Tachykinin A or fragments thereof of at least 5 amino acids with sepsis or septic shock, wherein an elevated level above a certain threshold is predictive of sepsis, severe sepsis or septic shock.

Substance P (SP) is a neuropeptide: an undecapeptide that functions as a neurotransmitter and as a neuromodulator. It belongs to the tachykinin neuropeptide family. SP is one of five members of the tachykinin family that includes neurokinin A, neuropeptide K, neuropeptide γ, and neurokinin B in addition to SP. They are produced from a protein precursor after differential splicing of the prepro-Tachykinin A gene (Helke et al. 1990. FASEB Journal 4(6):1606-15). SP plays a role in nociception, inflammation, plasma extravasation, platelet and leukocyte aggregation in post-capillary venules, and leukocyte chemotactic migration through vessel walls (Otsuka M, Yoshioka K. Neurotransmitter functions of mammalian tachykinins. Physiol Rev. 1993 April; 73(2):229-308). Substance P is produced by neuronal and non-neuronal cells, including immune cells (Bodkin and Fernandes 2012 Brit J Pharmacol 170:1279-1292).

The role of SP in sepsis remains unclear. On the one hand, in some studies it was found that SP could play a role in the inflammatory response to sepsis by the release of pro-inflammatory cytokines as interleukin (IL)-1, IL-6, and tumor necrosis factor (TNF)-alpha (Lotz et al. 1988. Science 241: 1218-1221; Laurenzi et al. 1990. Scand. J. Immunol. 31: 529-533; Ansel et al. 1993 J. Immunol. 150: 4478-4485; Yamaguchi et al. 2004. Inflamm. Res. 53: 199-204). On the other hand, in other studies it was found that SP could have anti-inflammatory effects by reducing TNF-alpha, IL-6, and inducible nitric oxide synthase (iNOS), and increasing IL-10 (Jiang et al. 2012. Neuroreport 23: 786-792; Jiang et al. 2013. Neuroreport 24: 846-851). Besides, according to the findings of further studies, SP could play a role in the microorganism clearance modulating the phagocytosis capacity (Verdrengh and Tarkowski 2008. Scand. J. Immunol. 67: 253-259; Yang et al. 2014. Crit. Care Med. 42: 2092-2100; Kincy-Cain and Bost 1996. J. Immunol. 157: 255-264; Lighvani et al. 2005. Eur. J. Immunol. 35: 1567-1575).

Circulating SP concentrations in septic patients have not been well studied. In one study with 61 septic patients, authors found higher serum SP concentrations in septic patients compared to healthy controls, and in non-survivor compared to survivor patients during the final phase of sepsis (Beer et al. 2002. Crit Care Med. 30:1794-1798). In another study with 42 septic patients were found lower plasma SP concentrations in septic patients compared to healthy controls (Arnalich et al. 1995. Life Sci 56: 75-81). In addition, serum SP levels were shown to be associated with mortality in sepsis (Lorente et al. 2015. J. Crit. Care 2015, 30, 924-928; Lorente et al. 2017. Int J Mol Sci 18(7): 1531). However, non-survivors showed lower serum SP levels compared to survivor patients (Lorente et al. 2015. J. Crit. Care 2015, 30, 924-928).

Moreover, in a mouse CLP-model, there was a time-dependent elevation of SP, the highest SP level was observed in plasma at the 1-h time point. The SP level was declined from the 5-h point onwards and peaked again at the 20-h point in plasma, demonstrating a biphasic response of substance P (Puneet et al. 2006. J Immunol 176: 3813-3820). In addition, administration of LPS to wild type mice caused a significant increase in circulating levels of SP (Ng et al. 2008. Journal of Leukocyte Biology 83: 288-295).

The potential roles for SP in sepsis are wide ranging (for review see Bodkin and Fernandes 2012 Brit J Pharmacol 170:1279-1292). It induces many inflammatory actions relevant to sepsis progression, most of which are attributed to activation of the NK1 receptor. SP is well known as an inducer of neurogenic inflammation, which shows hallmarks of vasodilation, oedema and leukocyte infiltration. All of these actions can be induced by SP acting on the NK1 receptor (O'Connor et al., 2004 J Cell Physiol 201:167-180), and are detrimental in sepsis. NK1 activation on endothelial cells induces cell retraction, leading to oedema, and also the production of vasodilators such as NO and prostacyclin (Katz et al., 2003. J Vet Pharmacol Ther 26: 361-368), which contribute to hypotension. NK1 activation can also induce inflammatory mediator transcription, including chemokines, cytokines and adhesion molecules in several cell types (Maggi, 1997. Regul Pept 70: 75-90). SP is also known to prime neutrophils for chemotactic responses to chemokines, inducing the expression of chemokine receptors, a response that can be inhibited with NK1 antagonists (Sun et al., 2007. Am J Physiol Cell Physiol 293: C696-C704). These actions of SP are detrimental during sepsis as they exacerbate the inflammation and lead to fatal organ damage. Oedema and vasodilation can contribute to the dangerous hypotension and decrease in lung function, which is associated with poor outcome from sepsis.

So far, investigations in humans have been hampered by the very short half-life of SP (12 min) (Conlon and Sheehan. Regul. Pept. 1983; 7:335-345). The recent development of an assay for a stable pro-Tachkinin A (PTA) fragment (N-terminal Pro-Tachykinin A or NT-PTA), which is a surrogate for labile SP (Ernst et al. Peptides 2008; 29: 1201-1206), has enabled studies on the role of this tachykinin system in human disease.

Delayed treatment in patients presenting to the emergency department (ED) with a suspected infection may result in a prolonged hospitalisation, an increased morbidity, and a greater rate of infection-related mortality. An accurate assessment of the severity of the host response and the potential for further disease progression to sepsis, severe sepsis, septic shock and organ dysfunction is therefore crucial in order to administer a rapid and targeted therapeutic response.

One object of the invention is therefore the use of pro-Tachykinin A or fragments thereof to distinguish patients who are more likely or have a high risk of developing sepsis, severe sepsis or septic shock requiring intensive treatments from patients who have a low risk of requiring such treatment.

It was the surprising finding of the present invention that pro-Tachykinin A or fragments thereof are early biomarker(s) for the prediction that a patient will later develop sepsis, severe sepsis and septic shock. The term “early biomarker” means that the level of the biomarker pro-Tachykinin A or fragments thereof is elevated in a patient before the patient has developed sepsis, severe sepsis or septic shock.

The term “subject” as used herein refers to a living human or non-human organism. Preferably herein the subject is a human subject. The subject may be healthy or diseased if not stated otherwise.

The term “elevated level” means a level above a certain threshold level.

A “bodily fluid” may be selected from the group comprising blood, serum, plasma, urine, cerebrospinal liquid (CSF), and saliva. A bodily fluid according to the present invention is in one particular embodiment a blood sample. A blood sample may be selected from the group comprising whole blood, serum and plasma. In a specific embodiment of the diagnostic method said sample is selected from the group comprising human citrate plasma, heparin plasma and EDTA plasma.

The term “prediction” relates to the prognosis of an outcome or a specific risk for a subject. This may also include an estimation of the chance of recovery or the chance of an adverse outcome for said subject.

The methods of the invention may also be used for monitoring, therapy monitoring, therapy guidance and/or therapy control. In the context of the present application “monitoring” relates to keeping track of a patient and potentially occurring complications, e.g., to analyze the progression of the healing process or the influence of a particular treatment or therapy on the health state of the patient.

The term “therapy monitoring” or “therapy control” in the context of the present invention refers to the monitoring and/or adjustment of a therapeutic treatment of said patient, for example by obtaining feedback on the efficacy of the therapy. As used herein, the term “therapy guidance” refers to application of certain therapies, therapeutic actions or medical interventions based on the value/level of one or more biomarkers and/or clinical parameter and/or clinical scores. This includes the adjustment of a therapy or the discontinuation of a therapy.

In the present invention, the terms “risk assessment” and “risk stratification” relate to the grouping of subjects into different risk groups according to their further prognosis. Risk assessment also relates to stratification for applying preventive and/or therapeutic measures. The term “therapy stratification” in particular relates to grouping or classifying patients into different groups, such as risk groups or therapy groups that receive certain differential therapeutic measures depending on their classification. The term “therapy stratification” also relates to grouping or classifying patients with infections or having symptoms of an infectious disease into a group that are not in need to receive certain therapeutic measures.

Sepsis is defined as life-threatening organ dysfunction caused by a dysregulated host response to infection (see Singer et al. 2016. JAMA 315(8): 801-810). Organ dysfunction can be identified as an acute change in total SOFA score≥2 points consequent to the infection. The baseline SOFA score can be assumed to be zero in patients not known to have preexisting organ dysfunction. A SOFA score≥2 reflects an overall mortality risk of approximately 10% in a general hospital population with suspected infection. Even patients presenting with modest dysfunction can deteriorate further, emphasizing the seriousness of this condition and the need for prompt and appropriate intervention, if not already being instituted. Sepsis is a life-threatening condition that arises when the body's response to an infection injures its own tissues and organs. Patients with suspected infection who are likely to have a prolonged ICU stay or to die in the hospital can be promptly identified at the bedside with qSOFA, i.e., alteration in mental status, systolic blood pressure≤100 mm Hg, or respiratory rate≥22/min.

Septic shock is a subset of sepsis in which underlying circulatory and cellular/metabolic abnormalities are profound enough to substantially increase mortality. Patients with septic shock can be identified with a clinical construct of sepsis with persisting hypotension requiring vasopressors to maintain mean arterial pressure (MAP)≥65 mm Hg and having a serum lactate level>2 mmol/L (18 mg/dL) despite adequate volume resuscitation. With these criteria, hospital mortality is in excess of 40%.

The term “sepsis” as used in the context of the present application relates to all possible stages in the development of sepsis.

The term “sepsis” also includes severe sepsis or septic shock based on the SEPSIS-2 definition (Bone et al. 1992. Crit Care Med 20(6):864-874). The term “sepsis” also includes subjects falling within the SEPSIS-3 definition (Singer et al. 2016 JAMA 315(8):801-810). As used herein, organ dysfunction denotes a condition or a state of health where an organ does not perform its expected function. “Organ failure” denotes an organ dysfunction to such a degree that normal homeostasis cannot be maintained without external clinical intervention. Said organ failure may pertain an organ selected from the group comprising kidney, liver, heart, lung, nervous system. By contrast, organ function represents the expected function of the respective organ within physiologic ranges. The person skilled in the art is aware of the respective function of an organ during medical examination.

Organ dysfunction may be defined by the sequential organ failure assessment score (SOFA-Score) or the components thereof. The SOFA score, previously known as the sepsis-related organ failure assessment score (Singer et al. 2016. JAMA 315(8): 801-10) is used to track a person's status during the stay in an intensive care unit (ICU) to determine the extent of a person's organ function or rate of failure. The score is based on six different scores, one each for the respiratory, cardiovascular, hepatic, coagulation, renal and neurological systems each scored from 0 to 4 with an increasing score reflecting worsening organ dysfunction. The criteria for assessment of the SOFA score are described for example in Lamden et al. (for review see Lambden et al. 2019. Critical Care 23: 374). SOFA score may traditionally be calculated on admission to ICU and at each 24-h period that follows. In particular, said organ dysfunction is selected from the group comprising renal decline, cardiac dysfunction, liver dysfunction or respiratory tract dysfunction.

The quick SOFA Score (quickSOFA or qSOFA) was introduced by the Sepsis-3 group in February 2016 as a simplified version of the SOFA Score as an initial way to identify patients at high risk for poor outcome with an infection (Angus et al. 2016. Critical Care Medicine. 44 (3): e113-e121). The qSOFA simplifies the SOFA score drastically by only including its 3 clinical criteria and by including “any altered mentation” instead of requiring a GCS<15. qSOFA can easily and quickly be repeated serially on patients. The score ranges from 0 to 3 points. One point is given for: low blood pressure (SBP≤100 mm Hg), high respiratory rate ((≥22 breaths/min) and altered mentation (GCS≤15). The presence of 2 or more qSOFA points near the onset of infection was associated with a greater risk of death or prolonged intensive care unit stay. These are outcomes that are more common in infected patients who may be septic than those with uncomplicated infection. Based upon these findings, the Third International Consensus Definitions for Sepsis recommends qSOFA as a simple prompt to identify infected patients outside the ICU who are likely to be septic (Seymour et al. 2016. JAMA 315(8): 762-774).

In preferred embodiments of the present invention the method is defined by the prediction of sepsis, severe sepsis and/or septic shock in a patient group, for whom it was previously difficult, if not impossible, to determine by routine clinical and/or molecular diagnostic means, whether a serious risk existed of serious deterioration of medical conditions, thereby requiring hospitalization. This patient group may be considered patients with mild symptoms of an infectious disease, or patients with no symptoms of serious infectious disease, e.g., without symptoms of sepsis.

In one embodiment, at the time of sample provision, the patient shows mild symptoms of an infectious disease corresponding to a qSOFA score of 0 or 1.

In one embodiment, at the time of sample provision, the patient shows no clinical symptoms for a sepsis, a severe sepsis and/or septic shock.

In one embodiment, at the time of sample provision, the patient has been diagnosed of having an infectious disease of bacterial, viral, fungal, or parasitic origin.

In one embodiment, at the time of sample provision, the patient has been diagnosed with community acquired pneumonia (CAP) or urinary tract infection (UTI).

It was entirely surprising that patients that only show mild symptoms of an infectious disease, such as patients corresponding to a qSOFA score of 0 or 1, and patients that show symptoms of an infectious disease that however do not indicate the presence of a sepsis, can be categorized as requiring hospitalization on the basis of determining a high-risk level of pro-Tachykinin A or fragments thereof according to the present invention. This represents a great advantage of the method of the invention, since a specialized physician would not expect, that patients with mild symptoms of an infectious disease and/or symptoms that do not hint towards sepsis would require hospitalization, for example to monitor progression of the symptoms of the infectious disease and perform treatment. Such patients may usually be examined by medical staff and subsequently released from continuous medical observation, for example with treatment instructions that can be carried out without professional medical supervision. However, the method of the present invention can identify patients, by objective means, who do not exhibit severe symptoms of sepsis and that still do require hospitalization.

In one embodiment of the invention, the method is used in order to stratify said patients into risk groups.

In one embodiment, a high-risk level of PTA or fragments thereof indicates that the patient is at risk of developing sepsis, severe sepsis and/or septic shock within 48 hours, 24 hours, 12 hours, 6 hours, 4 hours, preferably 2 hours and wherein a low-risk level of PTA or fragments thereof indicates that the patient is not at risk of developing sepsis, severe sepsis or septic shock within 48 hours, 24 hours, 12 hours, 6 hours, 4 hours, preferably 2 hours.

In one embodiment, the level of PTA or fragments thereof in said sample of bodily fluid is indicative of the risk of a development of a sepsis, severe sepsis and/or septic shock in the patient that requires hospitalization.

In one embodiment, a high-risk level of PTA or fragments thereof indicates that the patient is at risk of developing a sepsis, severe sepsis and/or septic shock that requires hospitalization within 48 hours, 24 hours, 12 hours, 6 hours, 4 hours, preferably 2 hours and wherein a low-risk level of PTA or fragments thereof indicates that the patient is not at risk of developing sepsis, severe sepsis and/or septic shock that requires hospitalization within 48 hours, 24 hours, 12 hours, 6 hours, 4 hours, preferably 2 hours.

In another embodiment of the invention, the method is used to stratify said patients into groups for early treatment (e.g., requirement of antibiotic administration). The term “early” is defined as time of treatment before the patient shows clinical signs and symptoms of sepsis, severe sepsis and/or septic shock or before the patient has been diagnosed for having sepsis, severe sepsis and/or septic shock.

In one embodiment, the level of PTA or fragments thereof in said sample of bodily fluid is indicative of the patient requiring frequent monitoring and/or critical care treatment. In one embodiment, the patient with a high-risk level will require a medical treatment provided in a hospital setting.

Examples of these treatments include but are not limited to fluid therapy, vasopressors, intravenous antibiotics, in some embodiments essentially any treatment beyond oral antibiotics, which may be self-administered at home.

Depending on the result of the method of the present invention, embodiments of the method may comprise subsequent therapeutic decisions and/or therapeutic actions. Such therapeutic decisions may include the initiation, change or modification of medical treatment. Preferably, if the method of the present invention is indicative of development to a condition requiring treatment in hospital, suitable therapeutic measures, such as initiation or change of a certain medication or fluid therapy may be initiated.

Any therapy, medical treatment or therapeutic action disclosed herein can be employed in the context of the method of the invention as a subsequent therapeutic decision or therapeutic action, in particular if the therapeutic measure is specifically administered in hospital, including but not limited to intravenous fluid therapy, dialysis, management of electrolyte abnormalities (in particular potassium, calcium and phosphorus). Furthermore, a maintained intensive observation and care of the patient may be indicated potentially over extended periods of time, such as several days, weeks or even months. This may involve keeping or moving the patient to an ICU and/or prolonging the stay of the patient in an ICU.

On the other hand, if the result of the method of the present invention is indicative of the absence of risk of developing a sepsis, severe sepsis or septic shock requiring hospitalization, no specific treatment measures with respect to such complication may be required, or less serious, self-administrable treatments may be prescribed.

Pro-Tachykinin A or fragments thereof are in particular for the before-mentioned medical utilities in the patient group of Emergency Department all-comers.

Throughout the specification the term Pro-Tachykinin and Pro-Tachykinin A (PTA) are used synonymously. The term includes all splice variants of Pro-Tachykinin A, namely αPTA, βPTA, γPTA, and δPTA. Throughout the specification it should be understood that the term fragments of Pro-Tachykinin A also include Substance P and Neurokinin A, Neuropeptide K, Neuropeptide γ, and Neurokinin B if not stated otherwise.

The term “determining the level of Pro-Tachykinin, its splice variants or fragments thereof of at least 5 amino acids including Substance P and Neurokinin” means, that usually the immunoreactivity towards a region within the before mentioned molecules is determined. This means that it is not necessary that a certain fragment is measured selectively. It is understood that a binder which is used for the determination of the level of Pro-Tachykinin or fragments thereof of at least 5 amino acids including Substance P and Neurokinin binds to any fragment that comprises the region of binding of said binder. Said binder may be an antibody or antibody fragment or a non-IgG scaffold.

Subject matter according to the present invention is a method, wherein the level of Pro-Tachykinin A or fragments thereof of at least 5 amino acids is determined by using a binder to Pro-Tachykinin A or fragments thereof of at least 5 amino acids.

In one embodiment of the invention said binder is selected from the group comprising an antibody, an antibody fragment or a non-Ig-scaffold binding to Pro-Tachykinin A or fragments thereof of at least 5 amino acids.

Alternative splicing of the PTA gene transcript generates four different PTA-mRNA molecules designated as αPTA, βPTA, γPTA, and δPTA, respectively (Harmar et al. 1990. FEBS Lett 275:22-4; Kawaguchi et al. 1986. Biochem Biophys Res Comm 139: 1040-6; Nawa et al. 1984. Nature 312:729-34), that differ in their exon combinations. All seven exons are solely contained in beta-PTA mRNA. However, the first three exons encoding for SP and a common N-terminal region consisting of 37 amino acids (SEQ ID NO. 5), are present in all PTA precursor molecules.

Alternative splicing gives the following Pro-Tachykinin A sequences:

(Isoform αPTA)
SEQ ID NO. 1
EEIGANDDLNYWSDWYDSDQIKEELPEPFEHLLQRIARRPKPQQFFGLM
GKRDADSSIEKQVALLKALYGHGQISHKMAYERSAMQNYERRR
(Isoform βPTA)
SEQ ID NO. 2
EEIGANDDLNYWSDWYDSDQIKEELPEPFEHLLQRIARRPKPQQFFGLM
GKRDADSSIEKQVALLKALYGHGQISHKRHKTDSFVGLMGKRALNSVAY
ERSAMQNYERRR
(Isoform γPTA)
SEQ ID NO. 3
EEIGANDDLNYWSDWYDSDQIKEELPEPFEHLLQRIARRPKPQQFFGLM
GKRDAGHGQISHKRHKTDSFVGLMGKRALNSVAYERSAMQNYERRRSEQ
(Isoform δPTA)
SEQ ID NO. 4
EEIGANDDLNYWSDWYDSDQIKEELPEPFEHLLQRIARRPKPQQFFGLM
GKRDAGHGQISHKMAYERSAMQNYERRR

Fragments of Pro-Tachykinin A that may be determined in a bodily fluid may be e.g., selected from the group of the following fragments:

Pro-Tachykinin A 1-37, P37, NT-PTA)
SEQ ID NO. 5
EEIGANDDLNYWSDWYDSDQIKEELPEPFEHLLQRIA
(Substance P)
SEQ ID NO. 6
RPKPQQFFGLM(-NH₂)
(Neuropeptide K)
SEQ ID NO. 7
DADSSIEKQVALLKALYGHGQISHKRHKTDSFVGLM(-NH₂)
(Neuropeptide Gamma)
SEQ ID NO. 8
GHGQISHKRHKTDSFVGLM(-NH₂)
(Neurokinin B)
SEQ ID NO. 9
HKTDSFVGLM(-NH₂)
(C-terminal flanking peptide, PTA 92-107)
SEQ ID NO. 10
ALNSVAYERSAMQNYE
(PTA 3-22)
SEQ ID NO. 11
GANDDLNYWSDWYDSDQIK
(PTA 21-36)
SEQ ID NO. 12
IKEELPEPFEHLLQRI

Determining the level of Pro-Tachykinin A or fragments thereof may mean that the immunoreactivity towards PTA or fragments thereof including Substance P and Neurokinin is determined. A binder used for determination of PTA or fragments thereof depending of the region of binding may bind to more than one of the above displayed molecules. This is clear to a person skilled in the art.

In a more specific embodiment of the invention fragments of PTA may be selected from SEQ ID NO. 5, SEQ ID NO. 10, SEQ ID NO. 11 and SEQ ID NO. 12.

In a more specific embodiment of the method according to the present invention the level of peptide 37 (P37), also termed PTA 1-37 or NT-PTA, SEQ ID NO. 5, is determined. In an even more specific embodiment according to the present invention at least one or two binders are used that bind to PTA 1-37 (NT-PTA), SEQ ID NO. 5, in case of more than one binder they bind preferably to two different regions within PTA 1-37 (NT-PTA), SEQ ID NO. 5. Said binder(s) may preferably be an antibody or a binding fragment thereof.

In an even more specific embodiment binder(s) are used for the determination of PTA, its variants and fragments that bind to one or both, respectively, of the following regions within PTA 1-37 (NT-PTA): PTA 3-22 (GANDDLNYWSDWYDSDQIK, which is SEQ ID NO. 11) and PTA 21-36 (IKEELPEPFEHLLQRI, which is SEQ ID NO. 12).

Thus, according to the present invention the level of immunoreactive analyte by using at least one binder that binds to a region within the amino acid sequence of any of the above peptide and peptide fragments, (i.e., Pro-Tachykinin A (PTA) and fragments according to any of the sequences 1 to 12), is determined in a bodily fluid obtained from said subject; and correlated to the specific embodiments of clinical relevance.

In a more specific embodiment of the method according to the present invention the level of PTA 1-37 is determined (SEQ ID NO. 5: NT-PTA).

In a more specific embodiment the level of immunoreactive analyte by using at least one binder that binds to Pro-Tachykinin A or fragments thereof of at least 5 amino acids is determined and is correlated to the above mentioned embodiments according to the invention to the specific embodiments of clinical relevance:
correlating said level of immunoreactive analyte with sepsis or septic shock, wherein an elevated level above a certain threshold is predictive of sepsis, severe sepsis or septic shock

Alternatively, the level of any of the above analytes may be determined by other analytical methods e.g., mass spectroscopy.

Subject matter of the present application is a method for predicting sepsis, severe sepsis and/or septic shock in a patient comprising:
determining the level of Pro-Tachykinin A or fragments thereof of at least 5 amino acids in a bodily fluid obtained from said subject, and
correlating the determined level of Pro-Tachykinin A or fragments thereof of at least 5 amino acids with sepsis or septic shock, wherein an elevated level above a certain threshold is predictive of sepsis, severe sepsis and/or septic shock.

One embodiment of the present application relates to a method for predicting sepsis, severe sepsis or septic shock in a patient, wherein the sample of bodily fluid of said patient is taken at a time where the patient shows no clinical symptoms for a sepsis, a severe sepsis and/or septic shock.

Another embodiment of the present application relates to a method for predicting sepsis, severe sepsis and/or septic shock in a patient, wherein said Pro-Tachykinin A is selected from the group comprising SEQ ID NO. 1 to 4 and fragments thereof are selected from the group comprising SEQ ID NO. 5 to 12.

Another embodiment of the present application relates to a method for predicting sepsis, severe sepsis and/or septic shock in a patient, wherein the level of Pro-Tachykinin A or fragments thereof of at least 5 amino acids is determined by using a binder to Pro-Tachykinin A or fragments thereof of at least 5 amino acids.

Another specific embodiment of the present application relates to a method for predicting sepsis, severe sepsis and/or septic shock in a patient, wherein the binder is selected from the group comprising an antibody, an antibody fragment or a non-Ig-Scaffold binding to Pro-Tachykinin A or fragments thereof of at least 5 amino acids.

Another preferred embodiment of the present application relates to a method for predicting sepsis, severe sepsis and/or septic shock in a patient, wherein said binder binds to a region within the amino acid sequence selected from the group comprising SEQ ID NO. 5, SEQ ID NO. 11 and SEQ ID NO. 12.

Another embodiment of the present application relates to a method for predicting sepsis, severe sepsis and/or septic shock in a patient, wherein the threshold range is between 75 and 200 pmol/L, more preferred between 90 and 175 pmol/L, even more preferred between 100 and 150 pmol/L, most preferred said threshold level is 120 pmol/L.

A further embodiment of the present application relates to a method for predicting sepsis, severe sepsis and/or septic shock in a patient, wherein the level of Pro-Tachykinin A is measured with an immunoassay and said binder is an antibody, or an antibody fragment binding to Pro-Tachykinin A or fragments thereof of at least 5 amino acids.

Another embodiment of the present application relates to a method for predicting sepsis, severe sepsis and/or septic shock in a patient, wherein an assay is used comprising two binders that bind to two different regions within the region of Pro-Tachykinin A that is amino acid 3-22 (SEQ ID NO. 11) and amino acid 21-36 (SEQ ID NO. 12), wherein each of said regions comprises at least 4 or 5 amino acids.

Another specific embodiment of the present application relates to a method for predicting sepsis, severe sepsis and/or septic shock in a patient, wherein an assay is used for determining the level of Pro-Tachykinin A or fragments thereof of at least 5 amino acids and wherein the assay sensitivity of said assay is able to quantify the Pro-Tachykinin A or Pro-Tachykinin A fragments of healthy subjects and is <10 pmol/L.

Another embodiment of the present application relates to a method for predicting sepsis, severe sepsis and/or septic shock in a patient, wherein said bodily fluid may be selected from the group comprising blood, serum, plasma, urine, cerebrospinal fluid (CSF), and saliva.

Another embodiment of the present application relates to a method for predicting sepsis, severe sepsis and/or septic shock in a patient, wherein additionally at least one biomarkers and/or clinical parameter and/or clinical scores is determined selected from the group comprising: D-Dimer, procalcitonin (PCT), C-reactive protein (CRP), lactate, penKid, ADM-NH₂, MR-proADM, NT-proBNP, BNP, presepsin, pentraxin-3 (PTX-3), CD-64, calprotectin, white blood cell count, lymphocyte count, neutrophil count, hemoglobin, platelet count, albumin, alanine transaminase, creatinine, blood urea, lactate dehydrogenase, creatinine kinase, cardiac troponin I, prothrombin time, serum ferritin, interleukin-6 (IL-6), IL-10, IL-2, IL-7, interferon gamma (IF-γ), tumor necrosis factor-α (TNF-α), granulocyte colony-stimulating factor (GCSF), IP-10, monocyte chemoattractant protein 1 (MCP-1), MIP-la, SOFA, qSOFA, APACHE II.

Another preferred embodiment of the present application relates to a method for predicting sepsis, severe sepsis and/or septic shock in a patient, wherein said determination is performed more than once in one patient.

One embodiment of the present application relates to a method for predicting sepsis, severe sepsis and/or septic shock in a patient in order to stratify said subjects into risk groups.

Subject matter of the present application is also a point-of-care (POC) device for performing a method for predicting sepsis, severe sepsis and/or septic shock in a patient, wherein said point of care device comprises at least two antibodies or antibody fragments directed to amino acid 3-22 (SEQ ID NO. 11) and amino acid 21-36 (SEQ ID NO. 12).

Subject matter of the present application is also a kit for performing a method for predicting sepsis, severe sepsis and/or septic shock in a patient, wherein said kit comprises at least two antibodies or antibody fragments directed to amino acid 3-22 (SEQ ID NO. 11) and amino acid 21-36 (SEQ ID NO.12).

Subject matter of the present application is also a method for assessing the risk of getting sepsis, severe sepsis and/or septic shock in a patient comprising:
determining the level of Pro-Tachykinin A or fragments thereof of at least 5 amino acids in a bodily fluid obtained from said subject, and
correlating the determined level of Pro-Tachykinin A or fragments thereof of at least 5 amino acids with sepsis or septic shock, wherein an elevated level above a certain threshold is indicative for the risk of the development of sepsis, severe sepsis and/or septic shock.

One embodiment of the present application relates to a method for assessing the risk of getting sepsis, severe sepsis and/or septic shock in a patient, wherein the sample of bodily fluid of said patient is taken at a time where the patient shows no clinical symptoms for a sepsis, a severe sepsis and/or septic shock.

Another embodiment of the present application relates to a method for assessing the risk of getting sepsis, severe sepsis and/or septic shock in a patient, wherein said Pro-Tachykinin A is selected from the group comprising SEQ ID NO. 1 to 4 and fragments thereof are selected from the group comprising SEQ ID NO. 5 to 12.

Another embodiment of the present application relates to a method for assessing the risk of getting sepsis, severe sepsis and/or septic shock in a patient, wherein the level of Pro-Tachykinin A or fragments thereof of at least 5 amino acids is determined by using a binder to Pro-Tachykinin A or fragments thereof of at least 5 amino acids.

Another specific embodiment of the present application relates to a method for assessing the risk of getting sepsis, severe sepsis and/or septic shock in a patient, wherein the binder is selected from the group comprising an antibody, an antibody fragment or a non-Ig-Scaffold binding to Pro-Tachykinin A or fragments thereof of at least 5 amino acids.

Another preferred embodiment of the present application relates to a method for assessing the risk of getting sepsis, severe sepsis and/or septic shock in a patient, wherein said binder binds to a region within the amino acid sequence selected from the group comprising SEQ ID NO. 5, SEQ ID NO. 11 and SEQ ID NO. 12.

Another embodiment of the present application relates to a method for assessing the risk of getting sepsis, severe sepsis and/or septic shock in a patient, wherein the threshold range is in the range between 75 and 200 pmol/L, more preferred between 90 and 175 pmol/L, even more preferred between 100 and 150 pmol/L, most preferred said threshold level is 120 pmol/L.

A further embodiment of the present application relates to a method for assessing the risk of getting sepsis, severe sepsis and/or septic shock in a patient, wherein the level of Pro-Tachykinin A is measured with an immunoassay and said binder is an antibody, or an antibody fragment binding to Pro-Tachykinin A or fragments thereof of at least 5 amino acids.

Another embodiment of the present application relates to a method for assessing the risk of getting sepsis, severe sepsis and/or septic shock in a patient, wherein an assay is used comprising two binders that bind to two different regions within the region of Pro-Tachykinin A that is amino acid 3-22 (SEQ ID NO. 11) and amino acid 21-36 (SEQ ID NO. 12), wherein each of said regions comprises at least 4 or 5 amino acids.

Another specific embodiment of the present application relates to a method for assessing the risk of getting sepsis, severe sepsis and/or septic shock in a patient, wherein an assay is used for determining the level of Pro-Tachykinin A or fragments thereof of at least 5 amino acids and wherein the assay sensitivity of said assay is able to quantify the Pro-Tachykinin A or Pro-Tachykinin A fragments of healthy subjects and is <10 pmol/L.

Another embodiment of the present application relates to a method for assessing the risk of getting sepsis, severe sepsis and/or septic shock in a patient, wherein said bodily fluid may be selected from the group comprising blood, serum, plasma, urine, cerebrospinal fluid (CSF), and saliva.

Another embodiment of the present application relates to a method for assessing the risk of getting sepsis, severe sepsis and/or septic shock in a patient, wherein additionally at least one biomarkers and/or clinical parameter and/or clinical scores is determined selected from the group comprising: D-Dimer, procalcitonin (PCT), C-reactive protein (CRP), lactate, penKid, ADM-NH₂, MR-proADM, NT-proBNP, BNP, presepsin, pentraxin-3 (PTX-3), CD-64, calprotectin, white blood cell count, lymphocyte count, neutrophil count, hemoglobin, platelet count, albumin, alanine transaminase, creatinine, blood urea, lactate dehydrogenase, creatinine kinase, cardiac troponin I, prothrombin time, serum ferritin, interleukin-6 (IL-6), IL-10, IL-2, IL-7, interferon gamma (IF-γ), tumor necrosis factor-α (TNF-α), granulocyte colony-stimulating factor (GCSF), IP-10, monocyte chemoattractant protein 1 (MCP-1), MIP-la, SOFA, qSOFA, APACHE II.

Another preferred embodiment of the present application relates to a method for assessing the risk of getting sepsis, severe sepsis and/or septic shock in a patient, wherein said determination is performed more than once in one patient.

One embodiment of the present application relates to a method for assessing the risk of getting sepsis, severe sepsis and/or septic shock in a patient in order to stratify said subjects into risk groups.

Subject matter of the present application is also a point-of-care device for performing a method for assessing the risk of getting sepsis, severe sepsis and/or septic shock in a patient, wherein said point of care device comprises at least two antibodies or antibody fragments directed to amino acid 3-22 (SEQ ID NO. 11) and amino acid 21-36 (SEQ ID NO. 12).

Subject matter of the present application is also a kit for performing a method for assessing the risk of getting sepsis, severe sepsis and/or septic shock in a patient, wherein said kit comprises at least two antibodies or antibody fragments directed to amino acid 3-22 (SEQ ID NO. 11) and amino acid 21-36 (SEQ ID NO.12).

Thus, subject matter of the present invention is method for assessing the risk of getting sepsis, severe sepsis and/or septic shock in a patient comprising:
determining the level of immunoreactive analyte by using at least one binder that binds to a region within the amino acid sequence of a peptide selected from the group comprising the peptides and fragments of SEQ ID NO. 1 to 12 in a bodily fluid obtained from said subject; and
   correlating said level of Pro-Tachykinin or fragments thereof with sepsis, severe sepsis and/or septic shock, wherein an elevated level above a certain threshold is indicative of the risk of development of sepsis, severe sepsis and/or septic shock. In a more specific embodiment of the present application the level of immunoreactive analyte by using at least one binder that binds to a region within the amino acid sequence of Pro-Tachykinin 1-37, N-terminal Pro-Tachykinin A fragment, NT-PTA (SEQ ID NO. 5) in a bodily fluid obtained from said subject.

In a specific embodiment of the present application the level of Pro-Tachykinin A or fragments thereof are measured with an immunoassay using antibodies or fragments of antibodies binding to Pro-Tachykinin A or fragments thereof. An immunoassay that may be useful for determining the level of Pro-Tachykinin A or fragments thereof of at least 5 amino acids may comprise the steps as outlined in Example 1. All thresholds and values have to be seen in correlation to the test and the calibration used according to Example 1. A person skilled in the art may know that the absolute value of a threshold might be influenced by the calibration used. This means that all values and thresholds given herein are to be understood in context of the calibration used herein (Example 1).

According to the invention the diagnostic binder to Pro-Tachykinin A is selected from the group consisting of antibodies e.g. IgG, a typical full-length immunoglobulin, or antibody fragments containing at least the F-variable domain of heavy and/or light chain as e.g. chemically coupled antibodies (fragment antigen binding) including but not limited to Fab-fragments including Fab minibodies, single chain Fab antibody, monovalent Fab antibody with epitope tags, e.g. Fab-V5Sx2; bivalent Fab (mini-antibody) dimerized with the CH3 domain; bivalent Fab or multivalent Fab, e.g. formed via multimerization with the aid of a heterologous domain, e.g. via dimerization of dHLX domains, e.g. Fab-dHLX-FSx2; F(ab′)2-fragments, scFv-fragments, multimerized multivalent or/and multispecific scFv-fragments, bivalent and/or bispecific diabodies, BITE® (bispecific T-cell engager), trifunctional antibodies, polyvalent antibodies, e.g. from a different class than G; single-domain antibodies, e.g. nanobodies derived from camelid or fish immunoglobulins.

In a specific embodiment of the present application the level of Pro-Tachykinin A or fragments thereof are measured with an assay using binders selected from the group comprising an antibody, an antibody fragment, aptamers, non-Ig scaffolds as described in greater detail below binding to Pro-Tachykinin A or fragments thereof.

Binder that may be used for determining the level of Pro-Tachykinin A or fragments thereof exhibit an affinity constant to Pro-Tachykinin A or fragments thereof of at least 10⁷ M⁻¹, preferred 10⁸ M⁻¹, preferred affinity constant is greater than 10⁹ M⁻¹, most preferred greater than 10¹⁰ M⁻¹ A person skilled in the art knows that it may be considered to compensate lower affinity by applying a higher dose of compounds and this measure would not lead out-of-the-scope of the invention. Binding affinity may be determined using the Biacore method, offered as service analysis e.g. at Biaffin, Kassel, Germany (http://www.biaffin.com/de/).

To determine the affinity of the antibodies, the kinetics of binding of PTA splice variants or fragments thereof to immobilized antibody was determined by means of label-free surface plasmon resonance using a Biacore 2000 system (GE Healthcare Europe GmbH, Freiburg, Germany).

Reversible immobilization of the antibodies was performed using an anti-mouse Fe antibody covalently coupled in high density to a CM5 sensor surface according to the manufacturer's instructions (mouse antibody capture kit; GE Healthcare). (Lorenz et al., “Functional Antibodies Targeting IsaA of Staphylococcus aureus Augment Host Immune Response and Open New Perspectives for Antibacterial Therapy”; Antimicrob Agents Chemother. 2011 January; 55(1): 165-173)

The assay may be calibrated by synthetic (for our experiments we used synthetic P37, SEQ ID NO. 5) or recombinant PTA splice variants or fragments thereof.

In addition to antibodies other biopolymer scaffolds are well known in the art to complex a target molecule and have been used for the generation of highly target specific biopolymers. Examples are aptamers, spiegelmers, anticalins and conotoxins. Non-Ig scaffolds may be protein scaffolds and may be used as antibody mimics as they are capable to bind to ligands or antigenes. Non-Ig scaffolds may be selected from the group comprising tetranectin-based non-Ig scaffolds (e.g. described in US 2010/0028995), fibronectin scaffolds (e.g. described in EP 1266 025; lipocalin-based scaffolds (e.g. described in WO 2011/154420); ubiquitin scaffolds (e.g. described in WO 2011/073214), transferring scaffolds (e.g. described in US 2004/0023334), protein A scaffolds (e.g. described in EP 2231860), ankyrin repeat based scaffolds (e.g. described in WO 2010/060748), microproteins preferably microproteins forming a cystine knot) scaffolds (e.g. described in EP 2314308), Fyn SH3 domain based scaffolds (e.g. described in WO 2011/023685) EGFR-A-domain based scaffolds (e.g. described in WO 2005/040229) and Kunitz domain based scaffolds (e.g. described in EP 1941867).

Another preferred embodiment of the present application relates to a method for predicting sepsis, severe sepsis and/or septic shock in a patient, wherein the threshold level of PTA or fragments thereof is between 75 and 200 pmol/L, more preferred between 90 and 175 pmol/L, even more preferred between 100 and 150 pmol/L, most preferred said threshold level is 120 pmol/L.

Another preferred embodiment of the present application relates to a method for assessing the risk of getting sepsis, severe sepsis and/or septic shock in a patient, wherein the threshold level of PTA or fragments thereof is between 75 and 200 pmol/L, more preferred between 90 and 175 pmol/L, even more preferred between 100 and 150 pmol/L, most preferred said threshold level is 120 pmol/L.

In another preferred embodiment the threshold for PTA or fragments thereof may be the upper normal range (99 percentile, 107 pmol/L).

In one specific embodiment of the present application the level of Pro-Tachykinin A or fragments thereof is measured with an immunoassay and said binder is an antibody, or an antibody fragment binding to Pro-Tachykinin A or fragments thereof of at least 5 amino acids.

In one specific embodiment of the present application the assay used comprises two binders that bind to two different regions within the region of Pro-Tachykinin A that is amino acid 3-22 (sequence, SEQ ID NO. 11) and amino acid 21-36 (sequence, SEQ ID NO. 12) wherein each of said regions comprises at least 4 or 5 amino acids.

In one embodiment of the present application the assays for determining Pro-Tachykinin A or Pro-Tachykinin A fragments in a sample of bodily fluid according to the present invention the assay sensitivity of said assay is able to quantify the Pro-Tachykinin A or Pro-Tachykinin A fragments of healthy subjects and is <20 pmol/, preferably <10 pmol/L and more preferably <5 pmol/L.

Subject matter of the present invention is the use of at least one binder that binds to a region within the amino acid sequence of a peptide selected from the group comprising the peptides and fragments of SEQ ID NO. 1 to 12 in a bodily fluid obtained from said subject in a method a for predicting sepsis, severe sepsis and/or septic shock in a patient.

Subject matter of the present invention is the use of at least one binder that binds to a region within the amino acid sequence of a peptide selected from the group comprising the peptides and fragments of SEQ ID NO. 1 to 12 in a bodily fluid obtained from said subject in a method a for assessing the risk of getting sepsis, severe sepsis and/or septic shock in a patient.

In one embodiment of the invention said binder is selected from the group comprising an antibody, an antibody fragment or a non-Ig-scaffold binding to Pro-Tachykinin A or fragments thereof of at least 5 amino acids. In a specific embodiment said at least one binder binds to a region with the sequences selected from the group comprising SEQ ID NO. 1, 2, 3, 4, 5, 6, 7, 8, 9 10, 11 and 12. In a specific embodiment said binder do not bind to SEQ ID NO. 6, 7, 8 and 9. In a specific embodiment said at least one binder binds to a region with the sequences selected from the group comprising SEQ ID No. 1, 2, 3, 4, 5, 11 and 12. In another specific embodiment said at least one binder binds to a region with the sequences selected from the group comprising SEQ ID No. 5, 11 and 12. In another very specific embodiment said binder bind to Pro-Tachykinin A 1-37, N-terminal Pro-Tachykinin A fragment, NT-PTA (SEQ ID NO. 5).

In a more specific embodiment of the present application the at least one binder binds to a region within the amino acid sequence of Pro-Tachykinin A 1-37, N-terminal Pro-Tachykinin A fragment, NT-PTA (SEQ ID NO. 5) in a bodily fluid obtained from said subject, more specifically to amino acid 3-22 (GANDDLNYWSDWYDSDQIK, SEQ ID NO. 11) and/or amino acid 21-36 (IKEELPEPFEHLLQRI, SEQ ID NO. 12), wherein each of said regions comprises at least 4 or 5 amino acids.

Thus, according to the present methods the level of immunoreactivity of the above binder is determined in a bodily fluid obtained from said subject. Level of immunoreactivity means the concentration of an analyte determined quantitatively, semi-quantitatively or qualitatively by a binding reaction of a binder to such analyte, where preferably the binder has an affinity constant for binding to the analyte of at least 10⁸ M⁻¹, and the binder may be an antibody or an antibody fragment or a non-IgG scaffold, and the binding reaction is an immunoassay.

Subject of the present invention is also a method for predicting sepsis, severe sepsis and/or septic shock in a patient according to any of the preceding embodiments, wherein the level of Pro-Tachykinin A or fragments thereof of at least 5 amino acids in a bodily fluid obtained from said subject either alone or in conjunction with other useful biomarkers and/or clinical parameter and/or clinical scores is used which may be selected from the following alternatives:
Comparison with the median of the level of Pro-Tachykinin A or fragments thereof of at least 5 amino acids in a bodily fluid obtained from said subject in an ensemble of pre-determined samples in a population of “healthy” or “apparently healthy” subjects,
Comparison with a quantile of the level of Pro-Tachykinin A or fragments thereof of at least 5 amino acids in a bodily fluid obtained from said subject in an ensemble of pre-determined samples in a population of “healthy” or “apparently healthy” subjects,
Calculation based on Cox Proportional Hazards analysis or by using Risk index calculations such as the NRI (Net Reclassification Index) or the IDI (Integrated Discrimination Index).

Subject of the present invention is also a method for assessing the risk of getting sepsis, severe sepsis and/or septic shock in a patient according to any of the preceding embodiments, wherein the level of Pro-Tachykinin A or fragments thereof of at least 5 amino acids in a bodily fluid obtained from said subject either alone or in conjunction with other useful biomarkers and/or clinical parameter and/or clinical scores is used which may be selected from the following alternatives:
Comparison with the median of the level of Pro-Tachykinin A or fragments thereof of at least 5 amino acids in a bodily fluid obtained from said subject in an ensemble of pre-determined samples in a population of “healthy” or “apparently healthy” subjects,
Comparison with a quantile of the level of Pro-Tachykinin A or fragments thereof of at least 5 amino acids in a bodily fluid obtained from said subject in an ensemble of pre-determined samples in a population of “healthy” or “apparently healthy” subjects,
Calculation based on Cox Proportional Hazards analysis or by using Risk index calculations such as the NRI (Net Reclassification Index) or the IDI (Integrated Discrimination Index).

Said additionally at least one biomarker and/or clinical parameter and/or clinical score may be determined selected from the group comprising: D-Dimer, procalcitonin (PCT), C-reactive protein (CRP), lactate, penKid, ADM-NH₂, MR-proADM, NT-proBNP, BNP, presepsin, pentraxin-3 (PTX-3), CD-64, calprotectin, white blood cell count, lymphocyte count, neutrophil count, hemoglobin, platelet count, albumin, alanine transaminase, creatinine, blood urea, lactate dehydrogenase, creatinine kinase, cardiac troponin I, prothrombin time, serum ferritin, interleukin-6 (IL-6), IL-10, IL-2, IL-7, interferon gamma (IF-γ), tumor necrosis factor-α (TNF-α), granulocyte colony-stimulating factor (GCSF), IP-10, monocyte chemoattractant protein 1 (MCP-1), MIP-la, SOFA, qSOFA, APACHE II.

Threshold levels can be obtained for instance from a Kaplan-Meier analysis, where the occurrence of a disease is correlated with the quartiles of the biomarker in the population. According to this analysis, subjects with biomarker levels above the 75th percentile have a significantly increased risk for getting the diseases according to the invention. This result is further supported by Cox regression analysis with full adjustment for classical risk factors: The highest quartile versus all other subjects is highly significantly associated with increased risk for getting a disease according to the invention.

Other preferred cut-off values are for instance the 90th, 95th or 99th percentile of a normal population. By using a higher percentile than the 75th percentile, one reduces the number of false positive subjects identified, but one might miss to identify subjects, who are at moderate, albeit still increased risk. Thus, one might adopt the cut-off value depending on whether it is considered more appropriate to identify most of the subjects at risk at the expense of also identifying “false positives”, or whether it is considered more appropriate to identify mainly the subjects at high risk at the expense of missing several subjects at moderate risk.

The above-mentioned threshold values might be different in other assays, if these have been calibrated differently from the assay system used in the present invention. Therefore, the above-mentioned threshold shall apply for such differently calibrated assays accordingly, taking into account the differences in calibration. One possibility of quantifying the difference in calibration is a method comparison analysis (correlation) of the assay in question (e.g., NT-PTA assay) with the respective biomarker assay used in the present invention by measuring the respective biomarker (e.g., NT-PTA) in samples using both methods. Another possibility is to determine with the assay in question, given this test has sufficient analytical sensitivity, the median biomarker level of a representative normal population, compare results with the mean biomarker levels (see example 2) and recalculate the calibration based on the difference obtained by this comparison. With the calibration used in the present invention, samples from normal (healthy) subjects have been measured: mean plasma NT-PTA was 55.2 pmol/L (SD+/−17.8 pmol/L).

A variety of immunoassays are known and may be used for the assays and methods of the present invention, these include: radio-immunoassays (“RIA”), homogeneous enzyme-multiplied immunoassays (“EMIT”), enzyme linked immunoadsorbent assays (“ELISA”), apoenzyme reactivation immunoassay (“ARIS”), chemiluminescence- and fluorescence-immunoassays, Luminex-based bead arrays, protein microarray assays, and rapid test formats such as for instance immunochromatographic strip tests (“dipstick immunoassays”) and immuno-chromatography assays.

In one embodiment of the invention such an assay is a sandwich immunoassay using any kind of detection technology including but not restricted to enzyme label, chemiluminescence label, electrochemiluminescence label, preferably a fully automated assay. In one embodiment of the invention such an assay is an enzyme labeled sandwich assay. Examples of automated or fully automated assay comprise assays that may be used for one of the following systems: Roche Elecsys®, Abbott Architect®, Siemens Centauer®, Brahms Kryptor®, Biomerieux Vidas®, Alere Triage®.

In one embodiment of the invention, it may be a so-called POC-test (point-of-care), that is a test technology which allows performing the test within less than 1 hour near the patient without the requirement of a fully automated assay system. One example for this technology is the immunochromatographic test technology.

In one embodiment of the invention at least one of said two binders is labeled in order to be detected.

In a preferred embodiment said label is selected from the group comprising chemiluminescent label, enzyme label, fluorescence label, radioiodine label.

The assays can be homogenous or heterogeneous assays, competitive and non-competitive assays.

In one embodiment, the assay is in the form of a sandwich assay, which is a non-competitive immunoassay, wherein the molecule to be detected and/or quantified is bound to a first antibody and to a second antibody. The first antibody may be bound to a solid phase, e.g., a bead, a surface of a well or other container, a chip or a strip, and the second antibody is an antibody which is labeled, e.g., with a dye, with a radioisotope, or a reactive or catalytically active moiety. The amount of labeled antibody bound to the analyte is then measured by an appropriate method. The general composition and procedures involved with “sandwich assays” are well-established and known to the skilled person (The Immunoassay Handbook, Ed. David Wild, Elsevier LTD, Oxford; 3rd ed. (May 2005), ISBN-13: 978-0080445267; Hultschig C et al., Curr Opin Chem Biol. 2006 February; 10(1):4-10. PMID: 16376134).

In another embodiment the assay comprises two capture molecules, preferably antibodies which are both present as dispersions in a liquid reaction mixture, wherein a first labelling component is attached to the first capture molecule, wherein said first labelling component is part of a labelling system based on fluorescence- or chemiluminescence-quenching or amplification, and a second labelling component of said marking system is attached to the second capture molecule, so that upon binding of both capture molecules to the analyte a measurable signal is generated that allows for the detection of the formed sandwich complexes in the solution comprising the sample.

In another embodiment, said labeling system comprises rare earth cryptates or rare earth chelates in combination with fluorescence dye or chemiluminescence dye, in particular a dye of the cyanine type.

In the context of the present invention, fluorescence based assays comprise the use of dyes, which may for instance be selected from the group comprising FAM (5-or 6-carboxyfluorescein), VIC, NED, Fluorescein, Fluoresceinisothiocyanate (FITC), IRD-700/800, Cyanine dyes, such as CY3, CY5, CY3.5, CY5.5, Cy7, Xanthen, 6-Carboxy-2′,4′,7′,4,7-hexachlorofluorescein (HEX), TET, 6-Carboxy-4′,5′-dichloro-2′,7′-dimethodyfluorescein (JOE), N,N,N′,N′-Tetramethyl-6-carboxyrhodamine (TAMRA), 6-Carboxy-X-rhodamine (ROX), 5-Carboxyrhodamine-6G (R6G5), 6-carboxyrhodamine-6G (RG6), Rhodamine, Rhodamine Green, Rhodamine Red, Rhodamine 110, BODIPY dyes, such as BODIPY TMR, Oregon Green, Coumarines such as Umbelliferone, Benzimides, such as Hoechst 33258; Phenanthridines, such as Texas Red, Yakima Yellow, Alexa Fluor, PET, Ethidiumbromide, Acridinium dyes, Carbazol dyes, Phenoxazine dyes, Porphyrine dyes, Polymethin dyes, and the like.

In the context of the present invention, chemiluminescence based assays comprise the use of dyes, based on the physical principles described for chemiluminescent materials in (Kirk-Othmer, Encyclopedia of chemical technology, 4th ed., executive editor, J. L Kroschwitz; editor, M. Howe-Grant, John Wiley & Sons, 1993, vol. 15, p. 518-562, incorporated herein by reference, including citations on pages 551-562). Preferred chemiluminescent dyes are acridiniumesters.

As mentioned herein, an “assay” or “diagnostic assay” can be of any type applied in the field of diagnostics. Such an assay may be based on the binding of an analyte to be detected to one or more capture probes with a certain affinity. Concerning the interaction between capture molecules and target molecules or molecules of interest, the affinity constant is preferably greater than 10⁸ M⁻¹.

In the context of the present invention, “binder molecules” are molecules which may be used to bind target molecules or molecules of interest, i.e., analytes (in the context of the present invention Pro-Tachyinin A and fragments thereof), from a sample. Binder molecules must thus be shaped adequately, both spatially and in terms of surface features, such as surface charge, hydrophobicity, hydrophilicity, presence or absence of lewis donors and/or acceptors, to specifically bind the target molecules or molecules of interest. Hereby, the binding may for instance be mediated by ionic, van-der-Waals, pi-pi, sigma-pi, hydrophobic or hydrogen bond interactions or a combination of two or more of the aforementioned interactions between the capture molecules and the target molecules or molecules of interest. In the context of the present invention, binder molecules may for instance be selected from the group comprising a nucleic acid molecule, a carbohydrate molecule, a PNA molecule, a protein, an antibody, a peptide or a glycoprotein. Preferably, the binder molecules are antibodies, including fragments thereof with sufficient affinity to a target or molecule of interest, and including recombinant antibodies or recombinant antibody fragments, as well as chemically and/or biochemically modified derivatives of said antibodies or fragments derived from the variant chain with a length of at least 12 amino acids thereof.

Chemiluminescent label may be acridinium ester label, steroid labels involving isoluminol labels and the like.

Enzyme labels may be lactate dehydrogenase (LDH), creatine kinase (CPK), alkaline phosphatase, aspartate aminotransferase (AST), alanine aminotransferase (ALT), acid phosphatase, glucose-6-phosphate dehydrogenase and so on.

In one embodiment of the invention at least one of said two binders is bound to a solid phase as magnetic particles, and polystyrene surfaces.

In one embodiment of the assays for determining Pro-Tachykinin A or fragments in a sample of bodily fluid according to the present invention such assay is a sandwich assay, preferably a fully automated assay. It may be an ELISA fully automated or manual. It may be a so-called POC-test (point-of-care). Examples of automated or fully automated assay comprise assays that may be used for one of the following systems: Roche Elecsys®, Abbott Architect®, Siemens Centauer®, Brahms Kryptor®, Biomerieux Vidas®, Alere Triage®. Examples of test formats are provided above.

In one embodiment of the assays for determining Pro-Tachykinin A or fragments in a sample of bodily fluid according to the present invention at least one of said two binders is labeled in order to be detected. Examples of labels are provided above.

In one embodiment of the assays for determining Pro-Tachykinin A or fragments in a sample of bodily fluid according to the present invention at least one of said two binders is bound to a solid phase. Examples of solid phases are provided above.

In one embodiment of the assays for determining Pro-Tachykinin A or fragments in a sample of bodily fluid according to the present invention said label is selected from the group comprising chemiluminescent label, enzyme label, fluorescence label, radioiodine label. A further subject of the present invention is a kit comprising an assay according to the present invention wherein the components of said assay may be comprised in one or more container.

In one embodiment subject matter of the present invention is a point-of-care device for performing a method according to the invention, wherein said point-of-care device comprises at least one antibody or antibody fragment directed to either amino acid 3-22 (GANDDLNYWSDWYDSDQIK, SEQ ID NO. 11) or amino acid 21-36 (IKEELPEPFEHLLQRI, SEQ ID NO. 12) wherein each of said regions comprises at least 4 or 5 amino acids.

In one embodiment subject matter of the present invention is a point-of-care device for performing a method according to the invention wherein said point-of-care device comprises at least two antibodies or antibody fragments directed to amino acid 3-22 (GANDDLNYWSDWYDSDQIK, SEQ ID NO. 11) and amino acid 21-36 (IKEELPEPFEHLLQRI, SEQ ID NO. 12), wherein each of said regions comprises at least 4 or 5 amino acids.

In one embodiment subject matter of the present invention is a kit or performing a method according to the invention, wherein said kit comprises at least one antibody or antibody fragment directed to either amino acid 3-22 (GANDDLNYWSDWYDSDQIK, SEQ ID No. 11) or amino acid 21-36 (IKEELPEPFEHLLQRI, SEQ ID NO. 12), wherein each of said regions comprises at least 4 or 5 amino acids.

In one embodiment subject matter of the present invention is a kit for performing a method according to the invention, wherein said kit comprises at least two antibodies or antibody fragments directed to amino acid 3-22 (GANDDLNYWSDWYDSDQIK, SEQ ID NO. 11) and amino acid 21-36 (IKEELPEPFEHLLQRI, SEQ ID NO. 12), wherein each of said regions comprises at least 4 or 5 amino acids.

The methods of the present invention may in part be computer-implemented. For example, the step of comparing the detected level of a marker, e.g., NT-PTA, with a reference and/or threshold level can be performed in a computer system. For example, the determined values may be entered (either manually by a health professional or automatically from the device(s) in which the respective marker level(s) has/have been determined) into the computer-system. The computer-system can be directly at the point-of-care (e.g., primary care unit or ED) or it can be at a remote location connected via a computer network (e.g., via the internet, or specialized medical cloud-systems, optionally combinable with other IT-systems or platforms such as hospital information systems (HIS)). Alternatively, or in addition, the associated therapy guidance and/or therapy stratification will be displayed and/or printed for the user (typically a health professional such as a physician).

The following embodiments are also subject of the present invention:
1. A method for predicting sepsis, severe sepsis and/or septic shock in a patient comprising:
   determining the level of Pro-Tachykinin A or fragments thereof of at least 5 amino acids in a sample of bodily fluid obtained from said subject, and
   correlating the determined level of Pro-Tachykinin A or fragments thereof of at least 5 amino acids with sepsis or septic shock, wherein an elevated level above a certain threshold is predictive of sepsis, severe sepsis and/or septic shock.
2. A method according to embodiment 1, wherein the sample of bodily fluid of said patient is taken at a time where the patient shows no clinical symptoms for a sepsis, a severe sepsis and/or septic shock or mild symptoms of an infectious disease.
3. A method according to embodiment 1 to 2, wherein said Pro-Tachykinin A is selected from the group comprising SEQ ID NO. 1 to 4 and fragments thereof are selected from the group comprising SEQ ID NO. 5 to 12.
4. A method according to embodiments 1 to 3, wherein the level of Pro-Tachykinin A or fragments thereof of at least 5 amino acids is determined by using a binder to Pro-Tachykinin A or fragments thereof of at least 5 amino acids.
5. A method according to embodiments 1 to 4, wherein the binder is selected from the group comprising an antibody, an antibody fragment or a non-Ig-scaffold binding to Pro-Tachykinin A or fragments thereof of at least 5 amino acids.
6. A method according to any of embodiments 1 to 5, wherein said binder binds to a region within the amino acid sequence selected from the group comprising SEQ ID NO. 5, SEQ ID NO. 11 and SEQ ID NO. 12.
7. A method according to any of the preceding embodiments, wherein the threshold range is between 75 and 200 pmol/L, more preferred between 90 and 175 pmol/L, even more preferred between 100 and 150 pmol/L, most preferred said threshold level is 120 pmol/L.
8. A method according to any of the preceding embodiments, wherein the level of Pro-Tachykinin A is measured with an immunoassay and said binder is an antibody, or an antibody fragment binding to Pro-Tachykinin A or fragments thereof of at least 5 amino acids.
9. A method according to any of the embodiments 1 to 8, wherein an assay is used comprising two binders that bind to two different regions within the region of Pro-Tachykinin A that is amino acid 3-22 (SEQ ID NO. 11) and amino acid 21-36 (SEQ ID NO. 12), wherein each of said regions comprises at least 4 or 5 amino acids.
10. A method according to any of embodiments 1 to 9, wherein an assay is used for determining the level of Pro-Tachykinin A or fragments thereof of at least 5 amino acids and wherein the assay sensitivity of said assay is able to quantify the Pro-Tachykinin A or Pro-Tachykinin A fragments of healthy subjects and is <10 pmol/L.
11. A method according to any of embodiments 1 to 10, wherein said bodily fluid may be selected from the group comprising blood, serum, plasma, urine, cerebrospinal fluid (CSF), and saliva.
12. A method according to embodiments 1 to 11, wherein additionally at least one biomarker and/or clinical parameter and/or clinical score may be determined selected from the group comprising: D-Dimer, procalcitonin (PCT), C-reactive protein (CRP), lactate, penKid, ADM-NH₂, MR-proADM, NT-proBNP, BNP, presepsin, pentraxin-3 (PTX-3), CD-64, calprotectin, white blood cell count, lymphocyte count, neutrophil count, hemoglobin, platelet count, albumin, alanine transaminase, creatinine, blood urea, lactate dehydrogenase, creatinine kinase, cardiac troponin I, prothrombin time, serum ferritin, interleukin-6 (IL-6), IL-10, IL-2, IL-7, interferon gamma (IF-γ), tumor necrosis factor-α (TNF-α), granulocyte colony-stimulating factor (GCSF), IP-10, monocyte chemoattractant protein 1 (MCP-1), MIP-la, SOFA, qSOFA, APACHE II.
13. A method according to any of embodiments 1 to 12, wherein said determination is performed more than once in one patient.
14. A method according to any of embodiments 1 to 13 in order to stratify said subjects into risk groups.
15. A point-of-care device for performing a method according to any of embodiments 1 to 14, wherein said point of care device comprises at least two antibodies or antibody fragments directed to amino acid 3-22 (SEQ ID NO. 11) and amino acid 21-36 (SEQ ID NO. 12).
16. A kit for performing a method according to any of embodiments 1 to 14, wherein said kit comprises at least two antibodies or antibody fragments directed to amino acid 3-22 (SEQ ID NO. 11) and amino acid 21-36 (SEQ ID NO.12).

### EXAMPLES

### Example 1—Development of Antibodies

### Peptides/Conjugates for Immunization

Peptides for immunization were synthesized (JPT Technologies, Berlin, Germany) with an additional N-terminal Cystein residue for conjugation of the peptides to bovine serum albumin (BSA). The peptides were covalently linked to BSA by using Sulfo-SMCC (Perbio-science, Bonn, Germany). The coupling procedure was performed according to the manual of Perbio.

TABLE 1

| Peptide for immunization | PTA Sequence |
|---|---|
| (C)GANDDLNYWSDWYDSDQIK | 3-22 (SEQ ID NO. 11) |
| (C)IKEELPEPFEHLLQRI | 21-36 (SEQ ID NO. 12) |

### Generation of Monoclonal Antibodies

A BALB/c mouse was immunized with 100 μg peptide-BSA-conjugate at day 0 and 14 (emulsified in 100 μl complete Freund's adjuvant) and 50 μg at day 21 and 28 (in 100 μl incomplete Freund's adjuvant). Three days before the fusion experiment was performed, the animal received 50 μg of the conjugate dissolved in 100 μl saline, given as one intraperitoneal and one intravenous injection. Splenocytes from the immunized mice and cells of the myeloma cell line SP2/0 were fused with 1 ml 50% polyethylene glycol for 30 s at 37° C. After washing, cells were seeded in 96-well cell culture plates. Hybrid clones were selected by growing in HAT medium (RPMI 1640 culture medium supplemented with 20% fetal calf serum and HAT-supplement). After two weeks the HAT medium is replaced with HT Medium for three passages followed by returning to the normal cell culture medium. The cell culture supernatants were primary screened for antigen specific IgG antibodies three weeks after fusion. The positive tested microcultures were transferred into 24-well plates for propagation. After re-testing the selected cultures were cloned and recloned using the limiting-dilution technique and the isotypes were determined (Lane, R. D. 1985: J. Immunol. Meth. 81: 223-228; Ziegler, B. et al. 1996: Horm. Metab. Res. 28: 11-15). Antibodies were produced via standard antibody production methods (Marx et al. 1997, Monoclonal Antibody Production ATLA 25, 121) and purified via Protein A-chromatography. The antibody purities were >95% based on SDS gel electrophoresis analysis.

### Labelling and Coating of Antibodies

Labelled compound (tracer, anti-PTA 3-22): 100 μg (100 μl) antibody (1 mg/ml in PBS, pH 7.4, was mixed with 10 μl Acridinium NHS-ester (1 mg/ml in acetonitrile, InVent GmbH, Germany) and incubated for 20 min at room temperature. Labelled antibody was purified by gel-filtration HPLC on Bio-Sil SEC 400-5 (Bio-Rad Laboratories, Inc., USA). Purified labelled antibody was diluted in (300 mmol/1 potassium-phosphate, 100 mmol/1 NaCl, 10 mmol/1 Na-EDTA, 5 g/l bovine serum albumin, pH 7.0). The final concentration was approx. 800.000 relative light units (RLU) of labelled compound (approx. 20 ng labelled antibody) per 200 μl. Acridiniumester chemiluminescence was measured by using an AutoLumat LB 953 (Berthold Technologies GmbH & Co. KG).

Coating (solid phase, anti PTA 22-36): Polystyrene tubes (Greiner Bio-One International AG, Austria) were coated (18 h at room temperature) with antibody (1.5 μg antibody/0.3 ml 100 mmol/1 NaCl, 50 mmol/1 Tris/HCl, pH 7.8). After blocking with 5% bovine serum albumin, the tubes were washed with PBS, pH 7.4 and vacuum dried.

### Pro-Tachykinin a Immunoassay and Calibration

50 μl of sample (or calibrator) was pipetted into coated tubes, after adding labelled antibody (200 ul), the tubes were incubated for 2 h at 18-25° C. Unbound tracer was removed by washing 5 times (each 1 ml) with washing solution (20 mmol/1 PBS, pH 7.4, 0.1% Triton X-100). Tube-bound labelled antibody was measured by using a Luminometer LB 953, Berthold, Germany. The assay was calibrated, using dilutions of synthetic P37, diluted in 20 mM K₂PO₄, 6 mM EDTA, 0.5% BSA, 50 μM Amastatin, 100 μM Leupeptin, pH 8.0. PTA control plasma is available at ICI-diagnostics, Berlin, Germany. FIG. 1 shows a typical PTA dose/signal curve.

The analytical assay sensitivity was (the median signal generated by 20 determinations of 0-calibrator (no addition of PTA)+2SD2 standard deviations (SD), the corresponding PTA concentration is calculated from a standard curve) 4.4 pmol/L.

### Example 2—NT-PTA in Healthy Subjects

EDTA-plasma samples from fasting healthy subjects (n=4435, average age 56 years) were measured using the NT-PTA assay. The mean value of NT-PTA in the population was 55.2 pmol/L, standard deviation+/−17.8 pmol/L, the lowest value was 9.07 pmol/L and the 99^{th} percentile was 107.6 pmol/L. All values were detectable with the assay, since the assay sensitivity was 4.4 pmol/L. The distribution of PTA values in healthy subjects is shown in FIG. 2.

### Example 3—Endotoxin Study in Humans

Thirty-two healthy male volunteers (mean (±SE) age 23.9 [±0.7 years] were admitted to a hospital clinical research unit. Screening tests were all normal. The participants were challenged at t=0 h with LPS (Escherichia coli lipopolysaccharide, 0311.H10:k) as a bolus intravenous injection at a dose of 4 ng/kg, which is a standardized dose. Blood was collected at intervals from 2 h before LPS injection to 24 h thereafter. Blood was immediately centrifuged (4° C., 10 min, 3000 rpm) and plasma was stored at −20° C. until assayed. The determination of different biomarkers of inflammations interleukin-6 (IL-6), tumor necrosis factor alpha (TNFα), and procalcitonin (PCT) as well as N-terminal Protachykinin A (NT-PTA) in the test persons treated with endotoxin, showed a time-dependent course of concentrations of the substances in blood (see FIG. 3).

First, an increase of TNFα after about 1 hour after injection of the endotoxin occurs as expected. Shortly thereafter an increase of cytotoxins IL-6 follows (about 1.5 hours after injection of the endotoxin). After 3 hours the concentrations of TNFα and IL-6 are decreasing, while now, surprisingly, an increase of A-peptide concentration occurs, which reaches the starting level after only about 7 hours. PCT shows an increase of concentration after 5 hours and steadily increases during further course. The secretion of NT-PTA is inducible by sole injection of endotoxin and is one event in the immune cascade between TNFα/IL-6 and PCT.

PCT is an established marker of sepsis diagnosis and displays the onset of sepsis in this study. However, the NT-PTA concentration is elevated approximately 3 hours before the sepsis biomarker PCT is elevated. Therefore, it can be concluded that NT-PTA is a marker for prediction of sepsis.

### Example 4—PTA for Prediction of Sepsis within 48 Hours in Patients with CAP

A cohort of 218 subjects hospitalized with community acquired pneumonia (CAP) was investigated. Main outcome was sepsis and severe sepsis, which were defined according to international consensus conference criteria (Singer et al. 2016. JAMA 315(8): 801-10). A total of 105 patients developed sepsis/severe sepsis within 48 hours. NT-PTA was measured in EDTA-plasma samples on admission. Patients that developed sepsis/severe sepsis within 48 hours after admission had significantly higher NT-PTA concentrations compared to patients that do not develop sepsis/severe sepsis (p=0.0007) (FIG. 4) with an AUC of 0.632 (p=0.08) (FIG. 5). Table 2 shows exemplary cut-off values with respective sensitivity and specificity.

TABLE 2
Exemplary cut-off values (CAP-study)

| Cut-off value | Sensitivity | Specificity |
|---|---|---|
| NT-PTA (in pmol/L) | (in %) | (in %) |
| 60.0 | 65.7 | 56.6 |
| 80.0 | 55.2 | 63.7 |
| 100.0 | 47.2 | 68.7 |
| 120.0 | 40.5 | 73.0 |
| 140.0 | 37.7 | 78.3 |

### Example 5—PTA in Patients with Suspicion of Sepsis

712 patients hospitalized with suspicion of sepsis were investigated. Inclusion criteria were age≥18 years, qSOFA at least 1 (GCS<15), breathing rate≥22/min, systolic blood pressure≤100 mmHg. 198 patients developed sepsis or septic shock after hospitalization. NT-proTA was measured in EDTA-plasma samples on admission (FIG. 6). Patients who developed sepsis had significantly higher NT-proTA concentrations (median 125.8 pmol/L [IQR 77.8-237.8]) than patients that did not develop sepsis (76.9 pmol/L [55.5-115.2]). Moreover, in patients (n=23) who further developed septic shock, NT-proTA levels were significantly elevated (209.2 pmol/L [118.7-402.3]) compared to patients not developing septic shock or developing sepsis. ROC-plot analysis revealed an AUC of 0.703 (p<0.0001) for NT-proTA to differentiate between patients who will develop sepsis or septic shock after admission from patients who will not develop sepsis or septic shock (FIG. 7).

Table 3 shows exemplary cut-off values with respective sensitivity and specificity to differentiate between patient groups (never develop sepsis/septic shock versus development of sepsis/septic shock).

TABLE 3
Exemplary cut-off values to differentiate
between patient groups (never develop sepsis/
septic shock versus development of sepsis/
septic shock).

| Cut-off value | | |
|---|---|---|
| NT-PTA | Sensitivity | Specificity |
| (in pmol/L) | (in %) | (in %) |
| 60.0 | 85.4 | 31.5 |
| 80.0 | 74.7 | 52.7 |
| 100.0 | 62.6 | 67.1 |
| 120.0 | 55.6 | 76.9 |
| 140.0 | 46.5 | 84.2 |

ROC-plot analysis to differentiate between patients who will develop septic shock after admission from patients who will not develop septic shock revealed an AUC of 0.757 (p<0.0001) for NT-proTA (FIG. 8).

Table 4 shows exemplary cut-off values with respective sensitivity and specificity to differentiate between patient groups (never develop septic shock versus development of septic shock).

TABLE 4
Exemplary cut-off values to differentiate
between patient groups (never develop septic
shock versus development of septic shock).

| Cut-off value | | |
|---|---|---|
| NT-PTA | Sensitivity | Specificity |
| (in pmol/L) | (in %) | (in %) |
| 60.0 | 91.3 | 27.4 |
| 80.0 | 87.0 | 46.2 |
| 100.0 | 78.3 | 60.1 |
| 120.0 | 73.9 | 69.2 |
| 140.0 | 65.2 | 77.1 |

### Example 6—PTA in Patients Admitted to the Emergency Department (ED)

This was a prospective, observational trial enrolling 97 patients consecutively admitted to the emergency department of Sant' Andrea Hospital in Rome for acute pathological conditions, and further hospitalization. For each enrolled patient clinical laboratory data and plasma NT-proTA values were collected at arrival. The patient's characteristics are summarized in table 5. A phone call 60-day follow-up was performed after hospital discharge.

TABLE 5
Patient's characteristics (ED trial)

| Variable | N = 97 |
|---|---|
| Age (years) | 76 +/− 12 |
| Gender (male) | 60% |
| 60-day survival rate | 81.4% |
| Final diagnosis | |
| AHF | 40.2% |
| Sepsis | 21.6% |
| Local infection | 18.6% |
| Gastrointestinal disorders | 10.3% |
| other | 9.3% |

The survival rate was 81.4% and events (death) occurred mainly in the first week after admission to the hospital. NT-proTA was measured on admission. We correlated the initial PTA value with the in-hospital mortality. NT-proTA is highly prognostic for outcome in hospitalized ED patients (AUC/C index 0.795; p<0.00001). FIG. 9 shows a Kaplan-Meier-Plot for survival of ED patients according to a) quartiles of PTA on admission and b) Cut-off of 100 pmol/L of NT-proTA on admission.

Procalcitonin was measured on admission and 24/48 and 72 hours thereafter. PCT-values were used as a surrogate for the diagnosis of sepsis. Sepsis categories are defined as follows: high-high (HH)—sepsis developed and not yet in remission, low-high (LH)—sepsis developing, high-low (HL)—sepsis in remission, low-low (LL)—no sepsis. The change was defined as follows: low-high (LH) if PCT t0<1 ng/mL and at t24-t72 max PCT value>1 ng/mL; high-low (HL) if PCT t0>1 ng/mL and at t24-t72 max PCT value<75% of t0; low-low (LL) if PCT t0<1 ng/mL at all time points; high-high (HH) if PCT t0>1 ng/mL at all time points. NT-proTA values for sepsis categories (within 72-hours, p=0.049) are shown in FIG. 10. NT-proTA concentrations in the respective subgroups are shown in table 6.

TABLE 6
NT-proTA levels in sepsis category subgroups

| Subgroup | Mean | SD | Median | IQR | Q1 | Q3 | Q.95 | Min | Max | n |
|---|---|---|---|---|---|---|---|---|---|---|
| high-high | 196.8 | 121.2 | 181.8 | 133.2 | 105.9 | 239.1 | 384.9 | 64.2 | 473.5 | 10 |
| high-low | 137.7 | 85.1 | 104.6 | 138.9 | 70.0 | 208.9 | 261.0 | 55.0 | 278.1 | 9 |
| low-high | 162.9 | 13.5 | 162.9 | 9.5 | 158.1 | 167.6 | 171.4 | 153.3 | 172.4 | 2 |
| low-low | 116.5 | 51.4 | 103.0 | 78.7 | 81.2 | 159.9 | 211.0 | 25.6 | 238.1 | 66 |

Patients that have sepsis and will not be on remission within the next 72 hours (HH) show higher NT-proTA values than patients that develop sepsis within 72 hours (LH), which are again higher than NT-proTA values in patients with sepsis in remission (HL) and patients developing not a sepsis (LL).

Table 7 shows the patient subgroups and the percentage of patients above exemplary cut-off values of 100, 120 and 140 pmol/L, respectively.

TABLE 7

| | % of patients | % of patients | % of patients |
|---|---|---|---|
| | (NT-proTA > | (NT-proTA > | (NT-proTA > |
| | 100 pmol/L | 120 pmol/L) | 140 pmol/L) |
| HH | 70% | 70% | 60% |
| HL | 56% | 44% | 44% |
| LH | 100% | 100% | 100% |
| LL | 56% | 38% | 29% |

### FIGURE DESCRIPTION

FIG. 1: A typical NT-proTachykinin A dose-signal curve.

FIG. 2: Frequency distribution of NT-ProTA in a healthy population (n=4463)

FIG. 3: Biomarkers of inflammation after treatment with endotoxin in plasma of healthy humans (IL-6, TNFα, PCT, NT-PTA)

FIG. 4: Box- and Whisker-Plot for NT-proTA values in CAP patients that did not develop sepsis/severe sepsis within 48 hours after admission versus CAP patients that develop sepsis/severe sepsis within 48 hours after admission (p=0.0007).

FIG. 5: ROC-plot for NT-proTA values in CAP patients that did not develop sepsis/severe sepsis within 48 hours after admission versus CAP patients that develop sepsis/severe sepsis within 48 hours after admission (AUC 0.632; p=0.08).

FIG. 6: Box- and Whisker-Plot for NT-proTA values on admission in hospitalized patients with suspicion of sepsis/septic shock.

FIG. 7: ROC-plot for NT-proTA values in patients who will develop sepsis or septic shock versus patients who will not develop sepsis or septic shock (AUC 0.703; p<0.0001).

FIG. 8: ROC-plot for NT-proTA values in patients who will develop septic shock after admission versus patients who will not develop septic shock (AUC 0.757; p<0.0001).

FIG. 9 a): Kaplan-Meier-Plot for survival of ED patients on admission (according to PTA quartiles)

FIG. 9 b): Kaplan-Meier-Plot for survival of ED patients on admission (PTA Cut-off 100 pmol/L)

FIG. 10: Median NT-proTA by change in sepsis categories

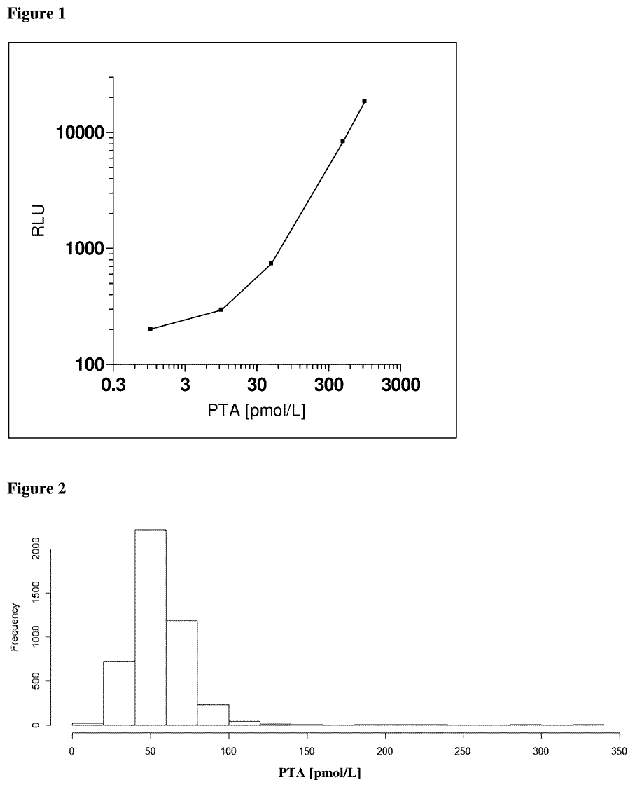

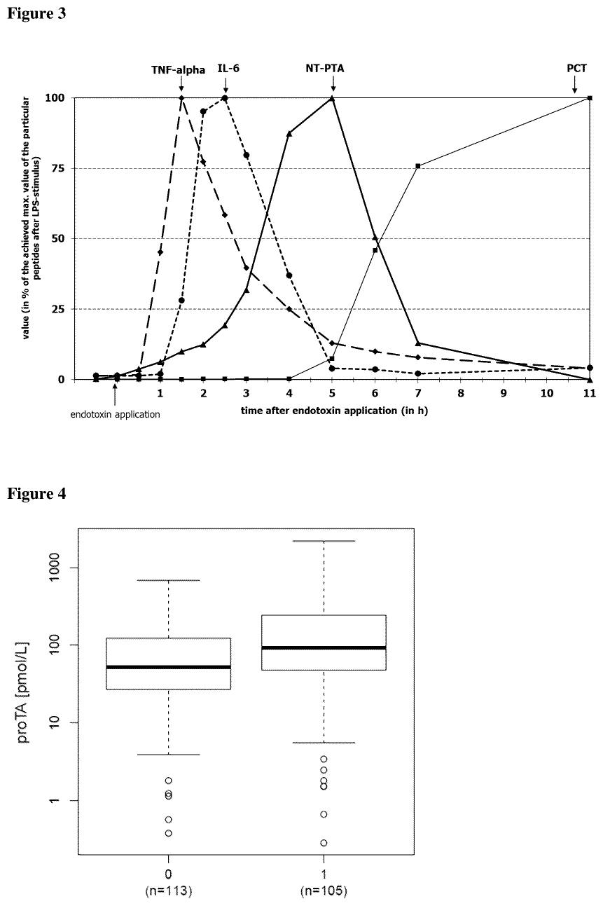

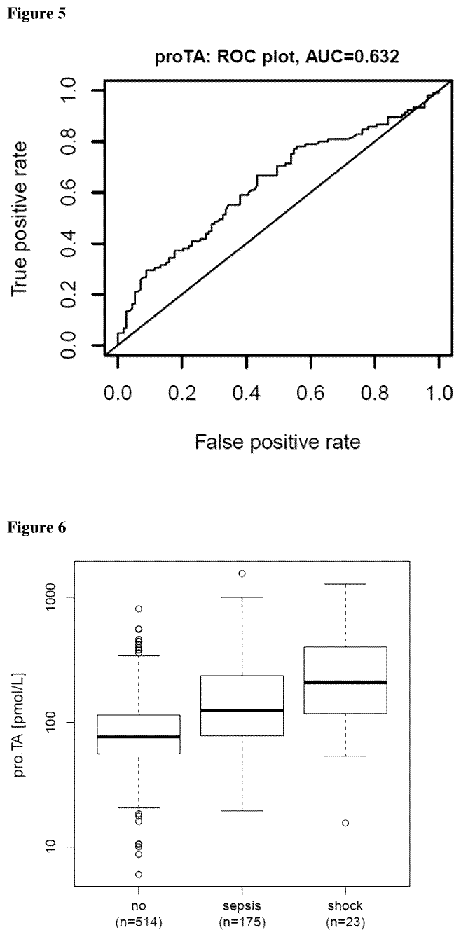

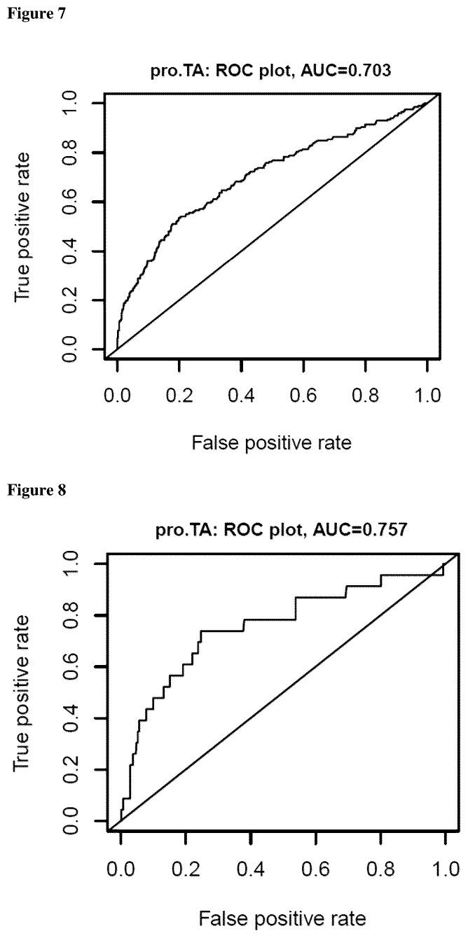

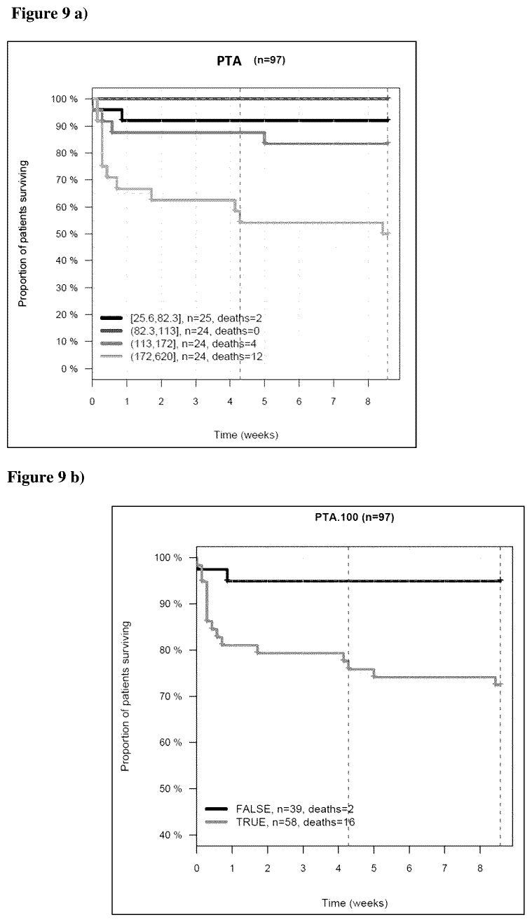

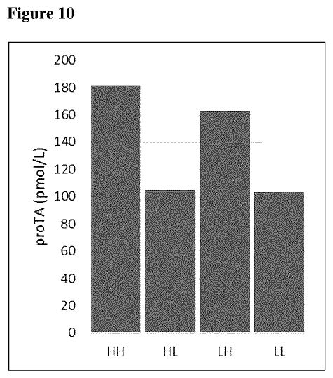

1. A method comprising:
receiving a sample comprising bodily fluid from a subject that has not been diagnosed with sepsis or septic shock and a binder obtained by a process comprising:
fusing splenocytes from a mammal previously immunized with a peptide-BSA-conjugate with cells of a myeloma cell line, and
screening for binders to Pro-Tachykinin A or fragments thereof of at least 5 amino acids; and
wherein the sample has at least 75 pmol/L of bound Pro-Tachykinin A or fragments thereof of at least 5 amino acids.

2. A method according to claim 1, wherein the sample of bodily fluid of said patient is taken at a time where the patient shows no clinical symptoms for a sepsis, a severe sepsis and/or septic shock or mild symptoms of an infectious disease.

3. A method according to claim 1, wherein said bound Pro-Tachykinin A is selected from the group comprising SEQ ID NO. 1 to 4 and fragments thereof are selected from the group comprising SEQ ID NO. 5 to 12.

4. (canceled)

5. (canceled)

6. A method according to claim 1, wherein said screening is conducted for binders to a region within an amino acid sequence selected from the group comprising SEQ ID NO. 5, SEQ ID NO. 11 and SEQ ID NO. 12.

7. A method according to claim 1, wherein is at least 120 pmol/L of bound Pro-Tachykinin A or fragments thereof of at least 5 amino acids is present in the sample.

8. A method according to claim 1, wherein the level of bound Pro-Tachykinin A is measured with an immunoassay and said binder is an antibody, or an antibody fragment binding to Pro-Tachykinin A or fragments thereof of at least 5 amino acids.

9. (canceled)

10. A method according to claim 1, wherein an assay is used to measure the level of bound Pro-Tachykinin A or fragments thereof of at least 5 amino acids and wherein the assay sensitivity of said assay is able to quantify the bound Pro-Tachykinin A or bound Pro-Tachykinin A fragments is <10 pmol/L.

11. A method according to claim 1, wherein said bodily fluid may be selected from the group comprising blood, serum, plasma, urine, cerebrospinal fluid (CSF), and saliva.

12. (canceled)

13. (canceled)

14. (canceled)

15. A point-of-care device for performing a method according to claim 1, wherein said point of care device comprises at least two antibodies or antibody fragments directed to amino acid 3-22 (SEQ ID NO. 11) and amino acid 21-36 (SEQ ID NO. 12).

16. A kit for performing a method according to claim 1, wherein said kit comprises at least two antibodies or antibody fragments directed to amino acid 3-22 (SEQ ID NO. 11) and amino acid 21-36 (SEQ ID NO.12).
